SEQUENCE LISTING (34 sequences)

SEQ ID NO: 1 - DNA, 24 bp, Artificial
  PCR Primer
ctgtgcctggtgcttcgttttgtg

SEQ ID NO: 2 - DNA, 23 bp, Artificial
  PCR Plasmid
caggacatagcgttggctacccg

SEQ ID NO: 3 - DNA, 26 bp, Artificial
  PCR Primer
cctcggcaattagcaacgtgaacatc

SEQ ID NO: 4 - DNA, 24 bp, Artificial
  PCR Primer
gtcctcgctcctcaggctctctcg

SEQ ID NO: 5 - DNA, 21 bp, Artificial
  PCR Primer
atgcgacgcaatcgtccgatc

SEQ ID NO: 6 - DNA, 24 bp, Artificial
  PCR Primer
ccctcctccaccaccactgctcat

SEQ ID NO: 7 - DNA, 22 bp, Artificial
  PCR Primer
ggagaagtgcctgcgccttgtg

SEQ ID NO: 8 - DNA, 25 bp, Artificial
  PCR Primer
agcttgaccctacgcccccaactga

SEQ ID NO: 9 - DNA, 23 bp, Artificial
  PCR Primer
tccaagtcgatggatatgcaacg

SEQ ID NO: 10 - DNA, 22 bp, Artificial
  PCR Primer
atgaatcgcaccgcatacactg

SEQ ID NO: 11 - DNA, 25 bp, Artificial
  PCR Primer
agctggtagccttaaaataagccaa

SEQ ID NO: 12 - DNA, 22 bp, Artificial
  PCR Primer
ttcctcgtgctttacggtatcg

SEQ ID NO: 13 - DNA, 21 bp, Artificial
  PCR Primer
gcagcccaagctgatcctcta

SEQ ID NO: 14 - DNA, 23 bp, Artificial
  PCR Primer
gcctgaaagaggtcatcatcacc

SEQ ID NO: 15 - DNA, 23 bp, Artificial
  PCR Primer
tttggtatttgagaaaggggctc

SEQ ID NO: 16 - DNA, 24 bp, Artificial
  PCR Primer
catctgcacgagactagtgagacg

SEQ ID NO: 17 - DNA, 26 bp, Artificial
  PCR Primer
gactacctcctgtacctgcaagccag

SEQ ID NO: 18 - DNA, 24 bp, Artificial
  PCR Primer
ctgccaatgtggatcagcattctc

SEQ ID NO: 19 - DNA, 25 bp, Artificial
  PCR Primer
cagcctgagcttcgacatcgtgaac

SEQ ID NO: 20 - DNA, 24 bp, Artificial
  PCR Primer
ctcaggaactcgtccaggtacacc

SEQ ID NO: 21 - DNA, 20 bp, Artificial
  PCR Primer
agcagagcgggtaaactggc

SEQ ID NO: 22 - DNA, 21 bp, Artificial
  PCR Primer
gacaatcggctgctctgatgc

SEQ ID NO: 23 - DNA, 25 bp, Artificial
  PCR Primer
aactaactctgtgttcagagccccg

SEQ ID NO: 24 - DNA, 20 bp, Artificial
  PCR Primer
tggctattgatttgggcagc

SEQ ID NO: 25 - DNA, 22 bp, Artificial
  PCR Primer
gcaatccaaaccaagcattgtc

SEQ ID NO: 26 - DNA, 20 bp, Artificial
  PCR Primer
tgacaccggcatttcgtcca

SEQ ID NO: 27 - DNA, 21 bp, Artificial
  PCR Primer
gcaaaaccaaattaagggcca

SEQ ID NO: 28 - DNA, 20 bp, Artificial
  PCR Primer
ttcccctgttcgcagttcaa

SEQ ID NO: 29 - DNA, 21 bp, Artificial
  PCR Primer
ccaacctgccatcacgagatt

SEQ ID NO: 30 - DNA, 21 bp, Artificial
  PCR Primer
ccaaagtcgccttcctcagaa

SEQ ID NO: 31 - DNA, 20 bp, Artificial
  PCR Primer
cttcctgtggggtttctttc

SEQ ID NO: 32 - DNA, 23 bp, Artificial
  PCR Primer
tacaaggttctgaagcaggtcca

SEQ ID NO: 33 - DNA, 24 bp, Artificial
  PCR Primer
ctcttgattcccactttgtggttc

SEQ ID NO: 34 - DNA, 22 bp, Artificial
  PCR Primer
gcgtttgagtttcgttttgtgc

(19) United States
(12) Patent Application Publication
(10) Pub. No.: US 2012/0107938 A1
(43) Pub. Date: May 3, 2012

(54) METHODS AND KITS FOR HIGH EFFICIENCY ENGINEERING OF CONDITIONAL MOUSE ALLELES

(75) Inventors: Javier Lopez-Rios, Basel (CH); Rolf Zeller, Basel (CH); Marco Osterrwalder, Basel (CH)

(73) Assignee: UNIVERSITY OF BASEL, Basel (CH)

(21) Appl. No.: 13/381,351

(22) PCT Filed: Jun. 28, 2010
(86) PCT No.: PCT/IB2010/001560

§ 371 Date: Dec. 28, 2011

Related U.S. Application Data
(60) Provisional application No. 61/221,144, filed on Jun. 29, 2009.

(51) Int. Cl. C12N 15/87; C12N 15/85

(52) U.S. Cl. 435/462; 435/320.1

(57) ABSTRACT

The present invention concerns methods and kits for the direct, targeted engineering of conditional alleles in rodent embryonic stem cells in which the conditional allele is replaced with a DNA of interest without first introducing heterotypic recombination sites, thus providing high efficiency targeted exchange of genetic material.

### FIELD OF THE INVENTION

The present invention relates to the field of molecular biology, in particular to recombinant engineering of eukaryotic cells, and more particularly to high efficiency targeted exchange of genetic material in conditional rodent alleles.

### BACKGROUND OF THE INVENTION

Various molecular biology strategies have been developed in order to better understand the function of genes in mice models, which are widely studied due to the similarity of the mouse and human genomes. Conventional Knock Out mice (“KO mice”), in which the activity of a gene is completely deleted, were the first models which provided valuable information about normal gene functioning. In this technique, both alleles of a gene are entirely knocked out so that the gene is completely absent from all cells. Newer technologies have since been developed in order to inactivate a gene only in a particular organ, cell type, or at a particular stage of development, which have led to the development of conditional KO Mice.

The conditional inactivation of a gene of interest was made possible by the advent in genetic engineering of special site-specific recombinases. These recombinase enzymes are able to specifically recognize genetic sequences referred to as Recombination Sites (“RS”) which flank a critical region of the gene of interest prior to its integration in the mouse's genome. The coding sequences of the recombinases can also be integrated into the mouse's genome and put under the control of one or more inducible promoters to regulate their activity. If the recombinase corresponding to the RS flanking the gene of interest is not expressed then the gene will function in a normal way; if the recombinase is expressed then the flanked region of gene is deleted and the phenotype of the deletion may be observed. Efforts in the art have been undertaken in order to build up huge libraries of conditional KO mice.

The generation of both conventional and conditional KO mice is achieved through Homologous Recombination (“HR”) in which genetic material is exchanged between two similar or identical strands of DNA. In this process, embryonic stem (“ES”) cells from mice may be transfected with a targeting vector carrying DNA sequences similar to that of the targeted gene, including regions that may be flanked with RSs. After recombination, the ES may be selected and injected into developing blastocysts, which are then implanted into an acceptor mother, which will in turn give birth to the modified progeny.

The process of developing genetic engineered mice is laborious and costly due both to the fragility of ES and to the low frequency of recombination events mediated by HR (0.1-10% depending on the targeted gene). Efforts to discover alternative methods for integrating genetic sequences in a targeted fashion have been unsuccessful.

However, it has been found that recombinases, previously used primarily to excise DNA sequences in conditional KO mice, may also be used in the exchange of DNA fragments. This discovery has been used to engineer mouse alleles to carry the elements needed by recombinases to mediate the exchange of a targeted gene with a gene of interest, rather than the excision of the targeted gene. Although the initial step of integrating the targeted gene with the elements necessary for the recombinases to mediate the exchange is still carried out by HR, further modification of the targeted gene may be achieved with recombinases rather than HR, resulting in an improved genetic platform for further modifications.

For DNA fragment exchange, “heterotypic” RSs are required (i.e., the RS flanking the target gene downstream must be different from the RS flanking the gene upstream). Heterotypic RSs which are still recognized by the respective recombinase have been developed and are used in Recombinase Mediated Cassette Exchange (“RMCE”), a recombination event which occurs much more frequently than HR (10-100% depending on the targeted gene) and thus results in lower costs associated with further modification of a particular gene (Soukharev et al. (1999) Nucleic Acids Res. 27, e21; Seibler, J. et al. (1998) J. Biochemistry 37, 6229-34).

Although RMCE is mediated by a single recombinase species, it has been shown that more than one distinct recombinase and respective RS may be employed for DNA exchange. For example, it has been shown that when a selection marker, flanked downstream by a first RS (e.g. FRT) for a first recombinase (e.g. Flp) and upstream by a second RS (e.g. loxP) for a second recombinase (e.g. Cre recombinase) is randomly integrated into the genome of wild-type ES, exchange of a genetic sequence carried on a targeting vector and flanked by the same RSs may be mediated by co-transfection and co-expression of the two respective recombinases with the targeting vector (Lauth et al. (2002) Nucleic Acids Res. 30, e115).

Despite these advances in the art, there has not been a technique developed for the direct manipulation of the genome of conditional knock-out mice, as current methods (such as RMCE) first require the introduction of heterotypic recombinase target sequences by classical homologous recombination, a costly, time-consuming, and inefficient step. Accordingly, there exist needs in the art for new techniques and related materials for custom engineering of the over 6500 presently available conditional mouse alleles (as well as future-developed conditional mouse alleles) with both homologous and heterologous modifications that can be introduced rapidly and highly efficiently. The present invention is directed to solving these and other needs.

### SUMMARY OF THE INVENTION

Generally speaking, the present invention addresses some or all of the above-described problems in the art by providing methods for the genetic engineering of conditional alleles in rodent embryonic stem cells, including: introducing into a rodent embryonic stem cell having a conditional allele that contains first and second recombination sites which are not identical and not recognized by the same recombinase

(a) a first and second recombinase specific for each of the first and second recombination sites, respectively; and
(b) a targeting vector that encodes a DNA sequence of interest flanked by the first and the second recombination sites, wherein the flanked region in the conditional allele is replaced with the sequence of interest; and further
(c) identifying and isolating embryonic rodent stem cells wherein the conditional allele is replaced with the DNA of interest.

In certain non-limiting embodiments the rodent embryonic stem cell is a mouse embryonic stem cell.

In certain non-limiting embodiments the first and the second recombinase are introduced in step (a) by introducing a fragment of DNA capable of expressing the first and the second recombinase, in particular by introducing a plasmid or plasmids capable of expressing the first and the second recombinase or by introducing a viral vector or viral vectors capable of expressing the first and the second recombinase; or by introducing purified recombinases that are able to be internalized by the cell.

In certain non-limiting embodiments identification in step (c) of correctly recombined embryonic rodent stem cells is based on properties of the replaced allele and/or the properties of the newly introduced DNA expression product. Preferred inventive methods comprise the step of screening the resulting cells and cell clones by PCR or other molecular biology techniques that reveal correctly recombined cell clones.

In certain non-limiting embodiments, the vector cassette in step (b) comprises a DNA encoding a selection marker and identification in step (c) is then accomplished by selecting transfected cells expressing said selection marker.

In certain non-limiting embodiments of the present invention, the plasmid used in the inventive methods is pDIRE.

In certain non-limiting embodiments of the present invention, each of the first and the second recombination sites used in the inventive methods is loxP and FRT.

In certain non-limiting embodiments of the present invention, each of the first and the second recombinases is CRE or FLP, or an active variant thereof.

In certain non-limiting embodiments of the present invention, each of the first and the second recombinases used in the inventive methods is iCRE or FLPo.

In certain non-limiting embodiments of the present invention, heterotypic recombination sites have not been introduced into the conditional allele prior to introduction of the targeting vector.

In certain non-limiting embodiments of the present invention, the targeting vector used in the inventive methods is generated using plasmids pDRAV-1, pDRAV-2, pDRAV-3 and pDRAV-4.

In certain non-limiting embodiments of the present invention, the targeting vector is transfected simultaneously with expression of the first and second recombinases.

In certain non-limiting embodiments of the present invention, the inventive methods are 10-70 fold more efficient than conventional homologous recombination methods.

In another aspect, the present invention includes a kit for the genetic engineering of conditional alleles in rodent embryonic stem cells, including: (a) a fragment of DNA, e.g. incorporated in a plasmid or plasmids, capable of expressing first and second recombinases specific for each of first and second recombination sites present in a conditional allele in a rodent embryonic stem cell to be replaced with a DNA sequence of interest; and (b) a targeting vector that encodes a DNA sequence of interest to replace the region in the conditional allele flanked by the first and the second recombination sites optionally including a selection marker.

In certain non-limiting embodiments of the present invention, each of the first and the second recombination sites on the targeting vector provided in the inventive kit is loxP or FRT.

In certain non-limiting embodiments of the present invention, each of the first and the second recombinases expressed by the plasmid provided in the inventive kit is CRE or FLP, or an active variant thereof.

In certain non-limiting embodiments of the present invention, each of the first and the second recombinases expressed by the plasmid provided in the inventive kit is iCRE or FLPo.

In certain non-limiting embodiments of the present invention, the inventive kit further includes plasmids pDRAV-1, pDRAV-2, pDRAV-3 and pDRAV-4 for use in generating the targeting vector.

### BRIEF DESCRIPTION OF THE FIGURES

FIG. 1 illustrates the inventive dRMCE technology strategy. The scheme of the target locus shows the configuration of a conditional mouse allele with a genomic region flanked by two loxP sites (arrow heads [symbol] and [symbol] ) and an outside selection cassette flanked by two FRT sites (triangles [symbol] and [symbol] ). Upon transfection, iCre/Flpo-mediated recombination in cis results in the deleted allele flanked by single loxP and FRT sites, which serves as a “docking site” for insertion of the replacement vector.

TL: Target locus; E: Exon; SC1: Selection cassette 1; DL: Deleted locus;
RV: Replacement vector; CM: Custom modification; SC2: Selection cassette 2;
RL: Replaced locus

FIG. 2 illustrates that dRMCE allows to efficiently modify difficult to target loci.

FIG. 2a: Targeting of the Hand2 locus by dRMCE. The available conditional Hand2 allele (Hand2^{f}) was used to insert a FLAG epitope tag into the Hand2 protein. The replacement vector was co-transfected into heterozygous Handt^{f/+} recipient mouse ES cells with the pDIRE plasmid. dRMCE-mediated correct replacement results in the Hand2^{FLAG} allele. The PGK-hygromycin selection cassette is flanked by the attB (slashed white rectangle) and attP (slashed black rectangle) target sites to enable its excision by the phiC31 recombinase. The relevant PCR primers (F, R) for colony screening are indicated. The EcoRV site is used to detect correct 5′ replacement by combining PCR amplification with an EcoRV restriction digestion.

TL: Target locus; E1: Exon 1 of Hand2; E2: Exon2 of Hand2; DL: Deleted locus; RV: Replacement vector; RL: Replaced locus; Hygro: Hygromycin resistance gene

FIG. 2b: PCR screening identified those Hand2 colonies with correct replacement at both ends (13%; lanes 1, 3 and 7). The scheme on the right shows the PCR fragment patterns indicative of a particular genomic configuration.

Col.: colony; Rec: recombination; Ctr.: control; TL: Target locus; DL: Deleted locus; RL: Replaced locus

FIG. 2c: Efficient germline transmission (lanes 1, 5, 6) of the Hand2^{FLAG} allele establishes that dRMCE does not affect the vigour of mouse ES cells.

F1: F1 progeny; mES: mouse embryonic stem cells

FIG. 3 illustrates plasmids and vectors used.

FIG. 3a shows a map of the pDIRE expression vector (Dual Improved Recombinase Expression). Simultaneous expression of both iCRE and FLPo recombinases in mouse ES cells is achieved by the use of heterologous promoters (PGK-FLPo; EF1α-iCre). pr: promoter.

FIG. 3b: The four pDRAV (Dual Recombinase Acceptor Vector) backbones containing the loxP and FRT sites in all possible orientations. A lox2272 site (white arrowhead [symbol] ) makes these replacement vectors compatible with conventional RMCE following dRMCE-mediated replacement of the conditional allele of choice. The polylinker in the pDRAV vectors provides the necessary versatility for rapid generation of custom-designed dRMCE replacement vectors.

Hygro: Hygromycin resistance gene

FIG. 3c: The pDREV (Dual Recombinase Eucomm-IKMC Vector) backbones in all three reading frames. The H2B-Venus coding sequence can be substituted by any cDNA of choice in a single cloning step.

SA: Splice acceptor; T: T2A encoding sequence; H2B-V: H2B-Venus fusion coding sequence; SpA: SV40pA sequence; puro: puromycin resistance cassette

FIG. 4 illustrates the inventive dRMCE technology proof of principle experimentation for the IKMC promoterless selection cassette alleles.

FIG. 4a: Schematic representation of the replacement in the Smad4 locus by dRMCE. The target locus is a Smad4 conditional allele (Smad4^{f}) with a promoterless selection cassette. This results in expression of a lacZ reporter and the neomycin resistance genes under control of the endogenous Smad4 promoter, which is active in embryonic stem cells. Co-transfection of the pDIRE and the pDREV-1 plasmids induces replacement via production of the Smade allele as intermediate. Correct trans-insertion of the replacement vector results in the Smad4^{YFP} allele. Note that the puromycin selection cassette is flanked by rox sites ( [symbol] ) to allow subsequent excision by the Dre recombinase.

Primers for PCR screening (F, R) are indicated.

TL: Target locus; E: Exon; SA: Splice acceptor; T: T2A encoding sequence; lacZ: beta-galactosidase gene; neo: neomycin resistance gene; DL: Deleted locus; RL: Replaced locus; H2B-V: H2B-Venus fusion coding sequence; puro: puromycin resistance cassette

FIG. 4b: Short-range PCR screening reveals a large number of clones with correct 3′ and 5′ replacement (69%, lanes 1, 3, 4, 5, 7, 8, 10, 11).

Col.: colony; Rec: recombination; 3′Rec, 5′Rec: correct replacement at the 3′ and 5′ end respectively; Wt: Wild-type mouse ES cells; Se: Smad4^{Δ} deleted locus; S4^{f}: Smad4 targeted locus.

FIG. 5: dRMCE works also efficiently with promoter-driven IKMC knockout-first alleles.

FIG. 5a: Scheme of the dRMCE strategy for the Zfp503 conditional allele (Zfp503^{f}), which contains three loxP sites (arrow head [symbol] ). The scheme illustrates the sequence of recombinase mediated cis deletion and trans insertion, which results in the correctly replaced Zfp503^{YFP} allele.

TL: Target locus; E: Exon; SA: Splice acceptor; I: IRES; T: T2A encoding sequence; lacZ: beta-galactosidase gene; neo: neomycin resistance gene; DL: Deleted locus; RL: Replaced locus; H2B-V: H2B-Venus fusion coding sequence; puro: puromycin resistance cassette

FIG. 5b: PCR screening reveals the high frequency of clones with correct replacement (lanes 1-3, 5 and 9) and some clones with only partial or no replacement. Col: colony; Rec: recombination; Ctr.: control; 3′Rec, 5′Rec: correct replacement at the 3′ and 5′ end respectively; Wt: Wild-type mouse ES cells.

FIG. 6 shows that random integration is neither detected for the targeting vector nor the pDIRE plasmid in mouse Hand2^{FLAG} ES cell clones.

FIG. 6a: Schematic view of the Hand2^{f} and Hand2^{FLAG} alleles. The positions of restriction sites and the probes used for Southern blot analysis are indicated.

TL: Target locus; E1: Exon 1 of Hand2; E2: Exon 2 of Hand2; neo: neomycin resistance gene; RL: Replaced locus; Hygro: Hygromycin resistance gene; 5′p: 5′ probe for Southern Blot; 3′p: 3′ probe for Southern Blot; hygro p: hygro probe for Southern Blot; H: HindIII; E: EcoRV; P: Pacl

FIG. 6b: Southern blot analysis confirms that replacement occurred correctly at both the 5′ (8.5 kb) and 3′ (6.9 kb) ends and reveals the integrity of the Hand2^{FLAG} locus. Col.: Colony; 5′p: 5′ probe for Southern Blot; 3′p: 3′ probe for Southern Blot

FIG. 6c: A single copy of the hygromycin-resistance cassette is present in all Hand2^{FLAG} mES cell clones.

Col.: Colony; hygro p: 5′ probe for Southern Blot

FIG. 6d: PCR primers that specifically amplify iCre and FLPo sequences fail to detect pDIRE sequences in Hand2^{FLAG} mES cell clones.

Col.: colony; Ctr.: control; Wt: Wild-type; N: Negative control

FIG. 7: Identification of correctly recombined colonies based on the use of reporters such as enzymatic activity or fluorescent proteins.

bgal: beta-galactosidase; pos: positive; neg: negative

### DETAILED DESCRIPTION OF THE INVENTION

Although gene targeting by homologous recombination (“HR”) in mouse embryonic stem (mES) cells is a powerful tool for tailored manipulation of the mouse genome, the frequencies of homologous recombination vary greatly between different loci (Capecchi, M. R. (1989) Science 244, 1288-92). In many cases, targeting frequencies by HR are rather low (e.g. less than 1%), which renders genetic manipulations time and cost intensive. In addition to constitutive mutations, conditional alleles can be generated by the introduction of loxP sites that permit tissue-specific or temporally controlled recombination by the CRE recombinase (Gu et al. (1994) Science 265, 103-6). These alleles are in general designed such that the selection cassette is flanked by FRT sites, which allows its removal using the FLP recombinase. Although additional flexibility is provided by recombinase-mediated cassette exchange (“RMCE”), which permits directed engineering of the region of interest (Branda, C. S. & Dymecki, S. M. (2004) Dev. Cell 6, 7-28; Wirth, D. et al. (2007) Curr. Opin. Biotechnol. 18, 411-9), RMCE requires prior introduction of heterotypic loxP or FRT sites into the locus of interest by HR, as discussed above.

The present invention is directed to new techniques, related materials and technologies (collectively referred to herein as “dual RMCE” and/or “dRMCE”) for the direct manipulation of the genome of conditional knock out mice or other rodents without first requiring the introduction of heterotypic recombinase target sequences by classical homologous recombination. As such, the present invention provides molecular biology tools that allows custom engineering of the over 6500 available conditional mouse alleles (as well as future-developed conditional mouse alleles and alleles in other non-human animals, e.g. rats) which cannot be directly manipulated with conventional techniques (including RMCE), which is possible due to the configuration in which RS sequences (e.g., loxP and FRT) were inserted into the genetic loci of the respective conditional alleles when they were initially generated.

Using a dRMCE plasmid toolkit of the present invention or alternative plasmids with similar characteristics, both homologous and heterologous modifications can be introduced rapidly and highly efficiently. For example, as recombination technologies require the work of dedicated and highly trained personnel for extended periods of time, the inventive dRMCE technologies greatly reduce costs, both with regard to human resources (employee salaries, etc.) as well as consumables (on the order of about 5-10 times less than those of conventional homologous recombination technologies) and, importantly, substantially reduce the period of time required from construct design to project completion (i.e., generation of ES and/or mice carrying the desired genetic modification). dRMCE has been found to offer high efficiency, with 10-69% of all clones correctly recombining.

As shown and discussed in detail herein, a non-limiting embodiment of the present invention, includes (1) an ES cell line that carries an allele containing RSs with the correct configuration of FRT and loxP sites; (2) a novel plasmid (“pDIRE”) that expresses both site-specific recombinases (iCRE and FLPo; improved versions of the conventional recombinases) or equivalent plasmids that separately encode CRE and FLP variants; and (3) a targeting vector that encodes the allele of interest flanked by a loxP and a FRT site with or without a selection marker (e.g. hygromycin drug resistance) to enable site-specific and oriented insertion into the genomic locus of interest. To facilitate the generation of the targeting vectors, in one embodiment of the present invention four acceptor plasmids (“pDRAV”), which greatly facilitate the generation of the custom-designed targeting vector, were generated. The set of generated plasmids accelerates the generation of the targeting vectors and minimizes the cloning steps by simplifying the invention procedure in certain embodiments. Furthermore, the materials (1)-(3) in the discussed embodiment may be provided in a kit for carrying out methods according to the present invention.

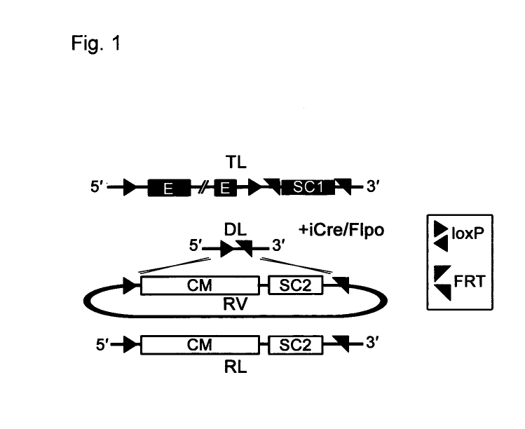

The inventive dRMCE technology utilizes the single loxP and FRT sites that remain in conditional loci upon CRE and FLP mediated recombination to enable reinsertion of sequences flanked by single loxP and FRT sites in a custom designed replacement vector (see FIG. 1), and are suited for the repeated manipulation of genomic loci that are difficult to target by HR and that contain multiple recombinase recognition sites, as is the case for most conditional alleles (minimally two loxP and FRT sites, see FIGS. 1, 2a, 4a, 5a and 6a).

Based on the procedures exemplified the invention more generally relates to a method for integrating a DNA of interest into a embryonic rodent stem cell having a conditional allele containing a first and a second recombination site, which are not identical and not recognized by the same recombinase, comprising

(a) introducing into said embryonic rodent stem cell a first and a second recombinase specific for the first and for the second recombination site, respectively;
(b) introducing into said embryonic rodent stem cell a targeting vector comprising a vector cassette that encodes said DNA of interest flanked by the first and the second recombination site, and
(c) identifying and isolating embryonic rodent stem cells wherein the conditional allele is replaced with the DNA of interest.

ES cells have been derived from several species, mainly from mice, but also from humans, rats and other species. It is therefore likely that similar extensive libraries of conditional alleles will be generated in the rat, and the inventive technology provided by dRMCE will be equally applicable to conditional alleles in such other species. The present invention is limited to non-human embryonic stem cells, such as rodent embryonic stem cells, in particular mouse embryonic stem cells.

Identification of correctly recombined cells, cell clones or cell colonies in step (c) is done by standard techniques well known in the art, i.e. molecular biology techniques and cellular biology techniques. Such methods may be based on properties of the replaced allele and/or the properties of the newly introduced DNA or DNA expression product. Preferably screening is done by PCR. In a particular embodiment the vector cassette in step (b) comprises a DNA encoding a selection marker, and identification in step (c) is done by selecting transfected cells expressing the selection marker.

Specifically, the first and the second recombinase are introduced in step (a) by introducing a fragment of DNA capable of expressing the first and the second recombinase. This may be done by introducing a plasmid or plasmids comprising such DNA capable of expressing the first and the second recombinase, or by introducing a viral vector or viral vectors capable of expressing the first and the second recombinase. Alternatively, purified recombinases that are able to be internalized by the cell may be introduced in step (a).

More specifically, the invention relates to a method for the genetic engineering of conditional alleles in mouse embryonic stem cells, including: introducing into a mouse embryonic stem cell having a conditional allele flanked with first and second recombination sites which are not identical (a) a plasmid capable of expressing first and second recombinases specific for each of the first and second recombination sites, respectively; and (b) a targeting vector that encodes a gene of interest flanked by the first and the second recombination sites and a selection marker, wherein the conditional allele is replaced with the gene of interest.

In particular, the recombination sites can be recombined by the CRE recombinase or an active variant thereof, and by the FLP recombinase or an active variant thereof, respectively. Examples of such recombination sites are loxP, lox71, lox66, lox511, lox5171, lox2272, lox2722, m2, L1 and loxN, which can be recombined by the CRE recombinase or an active variant thereof, and FRT, F3, F5, f2161, f2151, f2262, and f61, which can be recombined by the FLP recombinase or an active variant thereof.

Further considered are the recombinases Dre, phiC31 and phiBT1 and variants thereof, and corresponding recombination sites and variants thereof.

For selecting the embryonic rodent stem cells wherein the conditional allele is replaced with the DNA of interest, several methods are available. For example, selection may be performed based on antibiotic resistance incorporated by an antibiotic selection marker, for example the hygromycin resistance gene. Other antibiotic selection markers considered are those that confer resistance to drugs neomycin, puromycin, blasticidin, zeozin, mycophenolic acid, nourseothricin, actinomycin D, bleomycin sulfate, chlor-amphenicol, or mitomycin C. Selection may also be performed using hprt expression cassettes in HAT medium selection and hprt-deficient ES cells. Alternatively selection may be performed by using negative selection strategies such as those based on the use diphtheria toxin (dt)/DTA or DTR encoding cassettes, HSV-tk/ganciclovir/FIAU or hprt/6TG strategies. Alternatively, identification of correctly recombined colonies, clones or cells can be done by other means such as direct screening by PCR of individual clones or other molecular biology techniques such as Southern blot, Western blot, ELISA, immuno-histochemistry, FACS sorting or any other types of techniques that involve the use of antibodies to detect the presence or absence of a particular protein in the correctly recombined colonies or by using direct detection of the presence or absence of fluorescence, light or enzymatic activity in correctly recombined clones or by means that involve a growth advantage in correctly recombined clones.

Furthermore the invention relates to the use of the method as described herein for the generation of libraries of rodent, in particular mouse embryonic stem cells carrying single or multiple point mutations, single or multiple deletions or insertion of desired DNA into the locus of the conditional allele. DNA considered for integration into rodent embryonic stem cell is any type of DNA from the same or different species or artificially engineered, for example, DNA or genes encoding transcription factors, enzymes, structural proteins, adaptor proteins, extracellular proteins, membrane associated proteins, organelle-specific proteins, nuclear proteins, cytoplasmic proteins, secreted proteins, or genes encoding RNA other than mRNA. Alternatively, depending on the nature of the replaced allele, the DNA considered for integration into rodent stem cells is non-coding DNA that has gene or chromatin or chromosomal structural or regulatory functions of any kind.

The invention furthermore relates to kits comprising (a) a fragment of DNA, e.g. a plasmid or plasmids capable of expressing a first and a second recombinase specific for the first and for the second recombination site, respectively; and (b) a targeting vector comprising a vector cassette that encodes a DNA of interest flanked by the first and the second recombination site with or without a selection marker. The kits may contain further material customarily used in genetic engineering, such as restriction enzymes, DNA polymerases, purified Cre and/or Flp proteins or variants thereof, antibiotic(s) used for selection, and ES cell line suitable for dRMCE. In particular, the preferred kits comprise the plasmids indicated as preferred herein and exemplified, or variants thereof. The plasmid or plasmids capable of expressing the first and second recombinases could have other promoters, polyadenylation sequences, origin of replication or prokaryotic selection cassettes. The first and second recombinases expressed by the plasmid or plasmids or any fragment of DNA could be Cre or its variants, Flp or its variants, Dre or its variants, phiC31 or its variants or phiBT1 or its variants. The targeting vector could contain different variants of RS for the first and second recombinases. The region flanked by these RS could include different combinations of restriction sites for cloning and/or encode epitope or fluorescent tags, orthologous, paralogous and heterologous genes (human genes, other gene family members and unrelated genes such as recombinases, reporters and toxins). If containing a selection cassette, this could be of different types and could be or not be flanked by RS for other recombinases not taking part in the dual-RMCE experiment.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions and usages provided herein take precedent over any dictionary or extrinsic definition. That the present invention may be more readily understood, select terms are defined herein according to their usage.

In the context of the present invention “conditional allele” means any allele that carries two or more RS for a given recombinase and whose activity can be modified by the expression of that particular recombinase or its variants.

In the context of the present invention “active variant of the CRE recombinase” means any enzymatic activity that is able to mediate recombination between loxP target sites. Particular examples of such an active variant of the CRE recombinase are iCRE or any ligand-inducible CRE recombinases such as, CRE-ER^{T}, CRE-ER^{T2}, mCrem or Cre*PR.

In the context of the present invention “active variant of the FLP recombinase” means any enzymatic activity that is able to mediate recombination between FRT target sites. Particular examples of such an active variant of the FLP recombinase are FLPo, FLPe, FLP, FLP-L or FLP-ER.

The conditional mouse mutant alleles generated by the International Knockout Mouse Consortium (www.eucomm.org and www.komp.org; currently 6500 alleles available; Collins et al. (2007) Cell 129, 235) are compatible with the presently described and claimed dual RMCE but not with conventional RMCE. Finally, the dRMCE technology is also suited for engineering the conditional gene trap mouse ES cell-lines generated by the EUCOMM/IKMC consortium (currently 0.5700 available; Schnutgen et al. (2003) Nat. Biotechnol 21, 562-5).

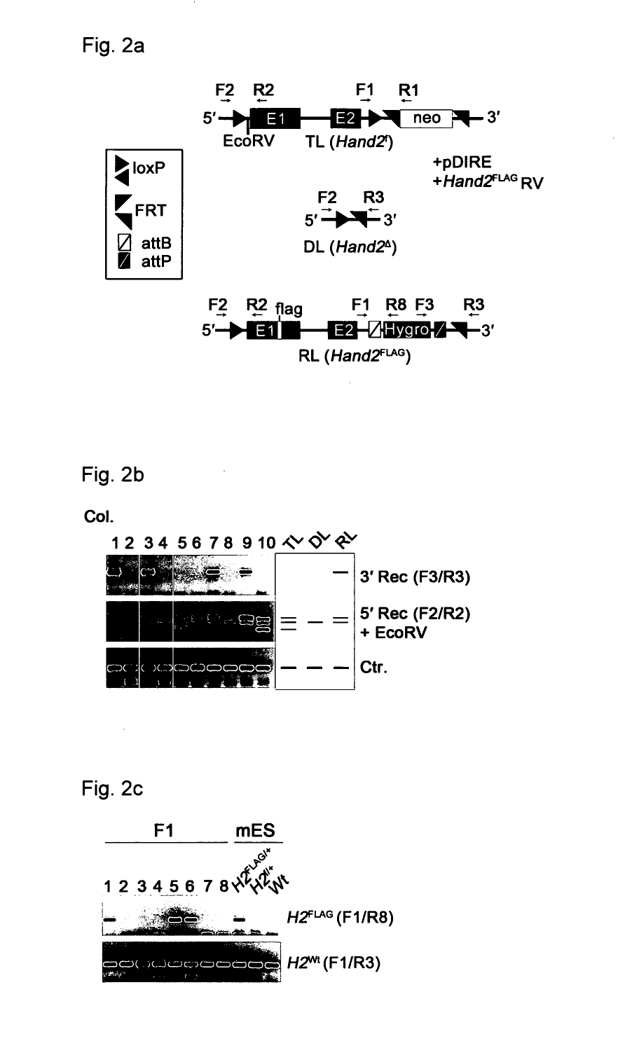

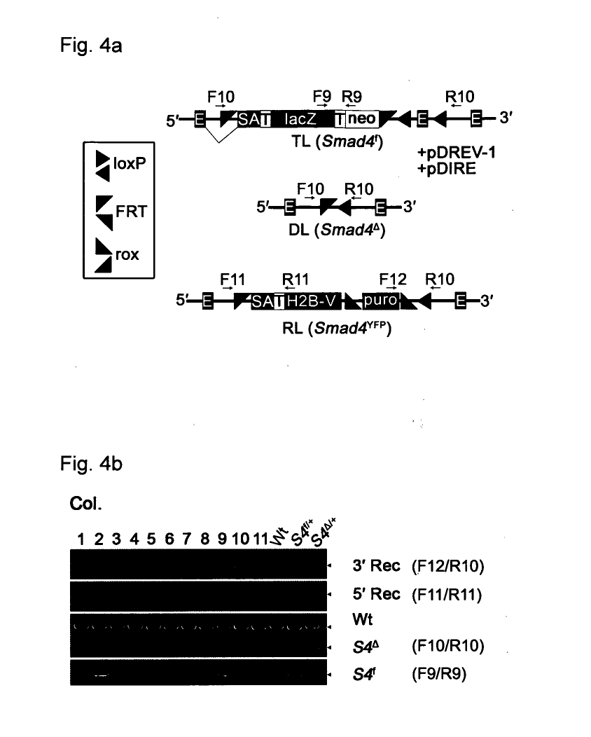

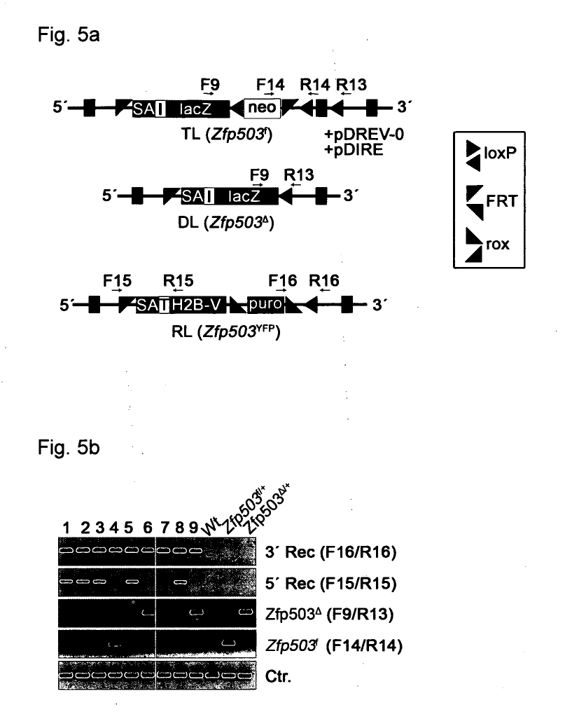

In general, dRMCE enables directed and highly efficient introduction of epitope (FIGS. 2a-2c) or fluorescent tags and/or single/multiple mutations into the endogenous gene products of interest. This is very important for e.g. in vivo biochemical studies and the generation of functional mouse ES cell and mouse models for human disease causing mutations. Furthermore, this technology permits the easy and rapid introduction of orthologous, paralogous and heterologous genes (human genes, other gene family members and unrelated genes such as recombinases, reporters (FIGS. 4a, 4b, 5a, 5b) and toxins) into any conditional allele of interest. The high efficiency of dRMCE makes this technology in principle suited for high throughput approaches that e.g. deal with functional screening of human disease-causing mutations in particular pathways or genes of interest.

As shown and described herein, and as will be evident to and appreciated by those of skill in the relevant art to which the present invention pertains, the present invention makes the generation of high throughput genetic and molecular assays based on manipulation of single or even multiple genes feasible and cost-efficient, as it eliminates the need to repeatedly re-target the locus by homologous recombination. Libraries of mouse ES that express epitope and/or fluorescently marked endogenous proteins or carry single/multiple point mutations/deletions/insertions and/or collections of genetically modified mice (carrying disease mutations in their endogenous loci, tagged proteins, orthologue or paralogue genes, toxin-encoding genes, reporter genes, CRE/FLP/DRE/phiC31/phiBT1 recombinases or its variants in specific cell populations) can be generated very fast using the inventive dRMCE technology, and such reagents/mouse strains are expected to be of significant value to applied research and/or the biotech sector. Use of methods and materials according to the present invention will generally avoid costly, time-consuming and inefficient repeated retargeting of the gene of interest by conventional HR. Moreover, the ease of cloning provided by the inventive dRMCE technology/toolkit and the fact that in most cases only 20-40 clones need to be picked, will permit research groups with limited experience and/or funds to engineer genes of interest starting with already available conditional alleles.

As mentioned, there are currently over 6500 alleles published that meet the criteria for the inventive dRMCE technology, most of which have been generated by research labs or international consortia and are readily available. Moreover, significant efforts are underway to develop additional mouse libraries (more than 15,000 are under construction). The inventive dRMCE technology is also useful, for example, with conditional gene trap mES lines (over 5700 of which have been identified as compatible with the inventive dRMCE technology). Other uses and application of the inventive dRMCE technology are contemplated herein and are within the scope of the present invention. For example, the modification/engineering of the genome in human, mouse or rat PS (induced pluripotent stem cells) or ES cells other than rodent and their re-implantation in the respective organism could be considered. Also, the inventive dRMCE technology may be applied to the recent development of Recombinase Mediated Genomic Replacement (“RMGR”) in which larger DNA fragments (for example, containing more than one allele) are exchanged using RMCE. The inventive dRMCE technology may be applied to the targeted modification of genomes by using naturally occurring CRE, FLP, DRE, phiC31 or phiBT1 recombination sites or pseudo-sites.

As mouse genetics is entering the age of systems biology and represents the major genetic animal model with direct relevance to human health and disease, improved technology rendering the thousands of conditional mouse alleles that have been generated over the years amenable to high efficient and straight forward further genetic engineering represents a major step forward. The inventive dRMCE technology provides exactly this and renders a great number of conditional alleles amenable to highly efficient, precise, straight forward and cost-effective further genetic engineering (10-69% frequency). Depending on the genomic region, the tested inventive dRMCE technology is 10-70 times more efficient than conventional homologous recombination of the same locus. This not only significantly reduces the time (3-6 months versus 6-12 months) and costs (5-10 times less), but provides a much more robust procedure than conventional homologous recombination, which can be very variable even for a particular locus and requires significant expert knowledge. Part of the time saving and rather easy use aspects are a consequence of the novel plasmid set generated in the present invention, which may be provided in a kit with other reagents for practicing the methods of the inventive dRMCE technology. The broad research applications for the inventive dRMCE technology will be readily apparent to those of skill in the relevant art.

Once given the above disclosure, many other features, modifications, and improvements will become apparent to the skilled artisan. Such features, modifications, and improvements are therefore considered to be part of this invention, without limitation imposed by the example embodiments described herein. Moreover, any word, term, phrase, feature, example, embodiment, or part or combination thereof, as used to describe or exemplify embodiments herein, unless unequivocally set forth as expressly uniquely defined or otherwise unequivocally set forth as limiting, is not intended to impart a narrowing scope to the invention in contravention of the ordinary meaning of the claim terms by which the scope of the patent property rights shall otherwise be determined.

The discussion herein and the following Example set forth various materials and methods used in the present invention and various embodiments of the present invention which are understood to be illustrative and non-limiting.

### EXAMPLES

### Construction of the pDIRE Expression Vector

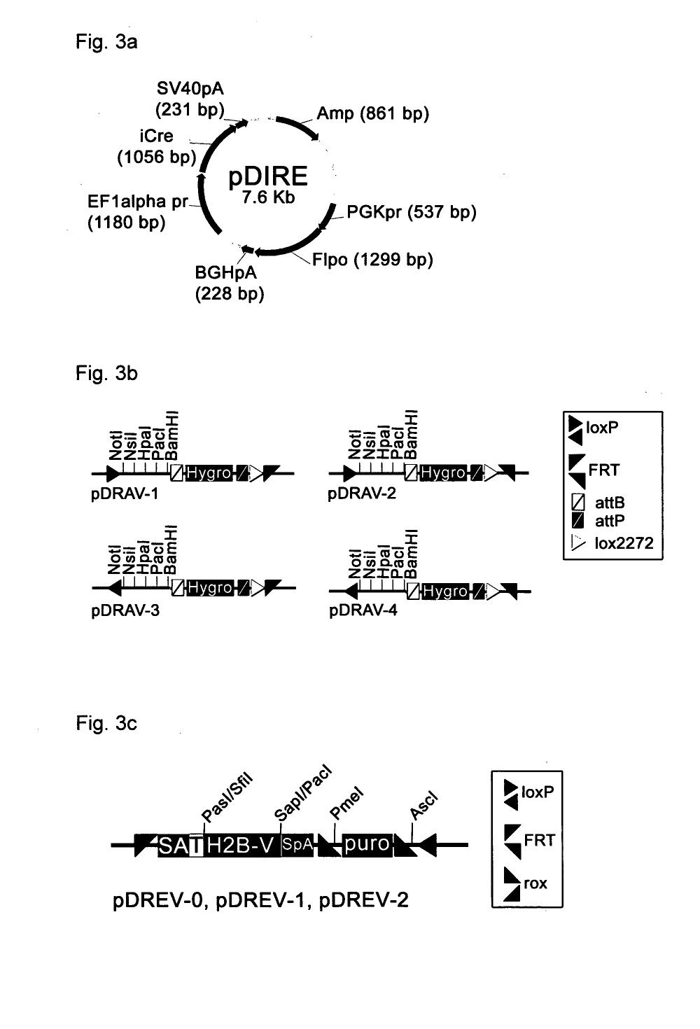

The iCre coding sequence was amplified by PCR from the pBOB-CAG-iCRE-SD plasmid (Addgene Plasmid 12336) using primers with specific restriction sites. Following SalI/Nott digestion, the iCre fragment was cloned into pBluescript II KS. Subsequently, the human EF1α promoter was inserted 5′ as a Hindlll-BamHl fragment derived from the BS513 EF1alpha-cre plasmid (Addgene Plasmid 11918). The SV40pA was inserted as a SpeI-SpeI fragment after PCR amplification from the pEGFP-N1 plasmid (GenBank Accession #U55762). These cloning steps resulted in the pEF1α-iCre cassette, which was completely sequenced. This iCre expression unit was isolated as a EcoRV-EcoRV fragment and inserted into the PsiI site of the pPGKFLPobpA plasmid (Addgene Plasmid 13793) to generate the pDIRE expression vector (FIG. 3a).

### Construction of the Hand2^{FLAG} Replacement Vector

Linkers were inserted into pBluescript II KS to produce the following restriction/recombinase site configuration: Sack/oxP-NarI-NotI-BamHI-SalI-Clal-FRTinv-HindIII-Kpnl. A NarI-NotI fragment of the Hand2 5′UTR and a NotI-BamHI fragment corresponding to the rest of the Hand2 transcription unit (with a FLAG-epitope tag inserted into coding exon 1, see below) were sequentially inserted into the pBluescript backbone. In the final step, a DNA fragment encoding the attB-pGK-Hygro-attP resistance cassette with 3′ HindIII and Pad sites for Southern blot screening was cloned into the BamHI/SalI sites of the pBluescript backbone, which results in the final replacement vector (FIG. 2a).

The Hand2 genomic locus was modified to introduce a loxP and EcoRV site into the NarI site 5′ of exon1. The second loxP site was inserted into the BamHI site 3′ of exon2 together with the selection cassette containing the pGK-Neo gene flanked by two FRT sites. The resulting Hand2 targeting vector was linearized with XhoI and introduced into mouse R1-ES cells. G418 resistant ES-cell clones (576 in total) were screened by Southern blot analysis. One ES-cell clone (4D7) was fully recombined and germ-line transmission from chimeric mice was obtained (Galli A. et al. (2010) PLoS Genet 6(4): e1000901).

Construction of the pDRAV Replacement Backbone Vectors

The four pDRAV vectors are shown in FIG. 3b. Similarly, the pBluescript II KS plasmid was modified by inserting linkers to produce all possible orientations of the loxP and FRT sites, as well as a lox2272 sequence that also enables conventional RMCE following initial replacement of a conditional allele by dRMCE. The attB-pGK-Hygro-attP resistance cassette was cloned into the BamHI-SalI sites. The multiple cloning sites of all pDRAV plasmids consist of unique NotI-NsiI-HpaI-Pact-BamHI restriction sites that can be used to insert the sequences of interest. HpaI, Pad and BamHI are well suited for restriction digests of genomic DNA for Southern analysis of mES cell clones.

Targeting of the Hand2 and Gli3 Loci by dRMCE

50 μg of the Hand2^{FLAG} (also designated Hand2^{FLAG-hygro} or Hand2^{FLAG-h}) replacement vector were co-electroporated with 50 μg of pDIRE into Hand2^{f/+} (also designated Hand2^{fneo/+}, (Galli A. et al. (2010) PLoS Genet 6(4): e1000901) or Gli3^{neo/+} R1-mES cells (1.5×10⁷ cells per cuvette; 240 kV and 475 μF), and plated in DMEM (4.5 g/l glucose) containing 15% FCS (HyClone), 2 mM D-glutamine, 1× non-essential amino acids, 2 mM sodium pyruvate, 1× penicillin/streptomycin, 0.1 mM β-mercaptoethanol and 10³ units/ml LIF/ESGRO (Chemicon; all other reagents from Gibco-Invitrogen). The culture medium was changed daily and from the second day onwards, resistant clones were selected in the presence of 175 μg/ml hygromycin (Sigma). After eight days in selection media, hygromycin resistant clones were picked and screened by PCR analysis. Positive clones were expanded, frozen in several aliquots and the correct replacement confirmed by detailed Southern Blot analysis.

Construction of the pDREV Replacement Vector Series.

A 1.75 Kb DNA fragment encoding the required elements (T2A/H2B-Venus/SV40 poly-adenylation sitelrox/Xhol/roxI/loxP) was synthesized and cloned as a BglII/HindIII fragment into the vector series L1L2-gt0/gt1/gt2 in all three possible reading frames. The PGK-puromycin selection cassette was excised as a SalI restriction fragment from the pPGKpuro plasmid and inserted into the Xhol site of the L1 L2-gt-H2B-Venus plasmid series. This resulted in the definitive pDREV replacement vector collection (pDREV-0, pDREV-1 and pDREV-2), which are compatible with all three open reading frames (FIG. 3c).

Targeting of the Smad4 and Zfp503 Loci by dRMCE

50 μg of the appropriate replacement vector were co-electroporated with 50 μg of pDIRE into mouse ES cells (1.5×10⁷ cells per cuvette; 240 kV and 475 pF). Smad4^{f}/+ or Zfp503^{f}/+ JM8 ES cells were grown in Knockout DMEM (4.5 g/l glucose) containing 10% FBS, 2 mM D-Glutamine, 1× Penicillin/Streptomycin, 0.1 m M beta-mercaptoethanol and 10³ units/ml LIF/ESGRO (Chemicon; all other reagents from Gibco-Invitrogen). The culture medium was changed daily and from the second day onwards, resistant colonies were selected in the presence of 175 μg/ml hygromycin or 0.5 μg/ml puromycin (Sigma). After eight days in selection media, drug-resistant colonies were picked and analysed by PCR.

Detection of dRMCE Replacement Events by PCR

The primer pairs used for PCR amplification are indicated in the corresponding figures. Their sequences and the sizes of all relevant amplicons are listed in Table 1. The screening strategy for the Hand2^{FLAG} allele is based on the loss of a single diagnostic EcoRV site in comparison to the Hand2^{f} allele due to the similar size of the 5′ PCR products. Amplification using the F2/R2 primer pair yields a double band at 435 by (Hand2^{FLAG} and Hand2^{f} alleles) and at 389 by (wild-type allele). This duplet is converted into a triplet in the Hand2^{f} allele by EcoRV digestion. The faint upper band remains, as the EcoRV digestion is partial in PCR buffer.

TABLE 1
Sequences of the PCR primers used and primer pairs/amplicon
sizes that detect the different configurations of the Hand2,
Gli3, Smad4 and Zfp503 loci following dRMCE

| Primer | Sequence | |
|---|---|---|
| F1 | CTGTGCCTGGTGCTTCGTTTTGTG | |
| R1 | CAGGACATAGCGTTGGCTACCCG | |
| F2 | CCTCGGCAATTAGCAACGTGAACATC | |
| R2 | GTCCTCGCTCCTCAGGCTCTCTCG | |
| F3 | ATGCGACGCAATCGTCCGATC | |
| R3 | CCCTCCTCCACCACCACTGCTCAT | |
| F4 | GGAGAAGTGCCTGCGCCTTGTG | |
| R4 | AGCTTGACCCTACGCCCCCAACTGA | |
| F5 | TCCAAGTCGATGGATATGCAACG | unrelated locus |
| R5 | ATGAATCGCACCGCATACACTG | (Grem1-control) |
| F6 | AGCTGGTAGCCTTAAAATAAGCCAA | |
| R6 | TTCCTCGTGCTTTACGGTATCG | |
| F7 | GCAGCCCAAGCTGATCCTCTA | |
| R7 | GCCTGAAAGAGGTCATCATCACC | |
| F8 | TTTGGTATTTGAGAAAGGGGCTC | |
| R8 | CATCTGCACGAGACTAGTGAGACG | |
| iCre-F | GACTACCTCCTGTACCTGCAAGCCAG | |
| iCre-R | CTGCCAATGTGGATCAGCATTCTC | |
| FLPo-F | CAGCCTGAGCTTCGACATCGTGAAC | |
| FLPo-R | CTCAGGAACTCGTCCAGGTACACC | |
| F9 | AGCAGAGCGGGTAAACTGGC | |
| R9 | GACAATCGGCTGCTCTGATGC | |
| F10 | AACTAACTCTGTGTTCAGAGCCCCG | |
| R10 | TGGCTATTGATTTGGGCAGC | |
| F11 | GCAATCCAAACCAAGCATTGTC | |
| R11 | TGACACCGGCATTTCGTCCA | |
| F12 | GCAAAACCAAATTAAGGGCCA | |
| R13 | TTCCCCTGTTCGCAGTTCAA | |
| F14 | CCAACCTGCCATCACGAGATT | |
| R14 | CCAAAGTCGCCTTCCTCAGAA | |
| F15 | CTTCCTGTGGGGTTTCTTTC | |
| R15 | TACAAGGTTCTGAAGCAGGTCCA | |
| F16 | CTCTTGATTCCCACTTTGTGGTTC | |
| R16 | GCGTTTGAGTTTCGTTTTGTGC | |

Primer pairs for screening: Hand2^{FLAG}

| Primer pair | Positive for: | Product size | Allele detected |
|---|---|---|---|
| F1/R1 | Hand2^{fneo} | 841 bp | 1 |
| F2/R2 | Hand2^{FLAG} (5′) | 435 bp + 389 bp | 7/8 |
| F3/R3 | Hand2^{FLAG} (′3) | 965 bp | 6/8 |
| F2/R3 | Hand2^{Δ} | 441 bp | 4 |

| | | |
|---|---|---|
| Primer pair | Positive for: | Product size |

Primer pairs for genotyping (Hand2^{FLAG} germline transmission):

| | | |
|---|---|---|
| F1/R8 | Hand2^{FLAG} | 404 bp |
| F1/R3 | Hand2 (wt) | 240 bp |

Primer pairs for screening: Gli3^{Hand2FLAG}

| | | |
|---|---|---|
| F3/R7 | Gli3^{Hand2 FLAG} (3′) | 1055 bp |
| F8/R2 | Gli3H^{and2 FLAG} (5′) | 613 bp |
| F7/R7 | Gli3^{Δneo} | 394 bp |
| F6/R6 | Gli3^{neo} | 1065 bp |

Primer pairs for screening: Smad4^{YFP}

| | | |
|---|---|---|
| F12/R10 | Smad4^{YFP}(3′) | 456 bp |
| F11/R11 | Smad4^{YFP}(5′) | 1594 bp |
| F10/R10 | Smad4^{wt} | 1265 bp |
| | Smad4^{Δ} | 565 bp |
| F9/R9 | Smad4^{f} | 558 bp |

Primer pairs for screening: Zfp503YFP

| | | |
|---|---|---|
| F16/R16 | Zfp503^{YFP}(3′) | 396 bp |
| F15/R15 | Zfp503^{YFP}(5′) | 1449 bp |
| F9/R13 | Zfp503^{Δ} | 987 bp |
| F14/R14 | Zfp503^{f} | 599 bp |

### Mice

All animal experiments were performed in accordance with Swiss law and have been approved by the regional veterinary authorities.

In Silico Data Mining for Conditional Alleles Compatible with dRMCE

The Mouse Genome Informatics database (www.informatics.jax.org) was interrogated for loxP/FRT sites containing conditional alleles, which were then individually analysed for their compatibility with dRMCE. The list of currently available compatible conditional alleles of mouse genes is included in Table 2.

TABLE 2
List of genes compatible with dRMCE including their
MGI database accession numbers and the PubMed Unique
Identifier (PMID), from the Mouse Genome Informatics
database (www.informatics.jax.org)

| Gene | Allele symbol | MGI number | PMID |
|---|---|---|---|
| Adm | Adm^{tm1Mtnz} | MGI:3811632 | 18723674 |
| Aifm1 | Aifm1^{tm2Pngr} | MGI:3686777 | 16287843 |
| Akap5 | Akap5^{tm1Jscoe} | MGI:3809936 | 18711127 |
| Akt2 | Akt2^{tm1Mbb} | MGI:2158455 | 11387480 |
| Apba1 | Apba1^{tm1Sud} | MGI:3697697 | 12547917 |
| Apba2 | Apba2^{tm1Sud} | MGI:3697709 | 17167098 |
| Apba3 | Apba3^{tm1Sud} | MGI:3697711 | 17167098 |
| Apc | Apc^{tm2Rak} | MGI:3688435 | 17002498 |
| Bambi | Bambi^{tm1Jian} | MGI:3758816 | 17661381 |
| Bdnf | Bdnf^{tm1Krj} | MGI:3582638 | 12890780 |
| Bhlhe40 | Bhlhe40^{tm1Rhli} | MGI:3775802 | 18234890 |
| Birc5 | Birc5^{tm1Mak} | MGI:3046203 | 14757745 |
| Bmp2 | Bmp2^{tm1Jfm} | MGI:3583785 | 15986484 |
| Bmp4 | Bmp4^{tm1Jfm} | MGI:3041440 | 15070745 |
| Bmp4 | Bmp4^{tm3.1Blh} | MGI:2181190 | 11857779 |
| Bmp4 | Bmp4^{tm4Blh} | MGI:3797048 | 18404215 |
| Braf | Braf^{tm1Wds} | MGI:3711006 | 17396120 |
| Cacna1g | Cacna1g^{tm1Stl} | MGI:3530499 | 15677322 |
| Card6 | Card6^{tm1Aldu} | MGI:3776907 | 18160713 |
| Cdc73 | Cdc73^{tm1Btt} | MGI:3794030 | 18212049 |
| Cdh2 | Cdh2^{tm1Glr} | MGI:3522469 | 15662031 |
| Cdh22 | Cdh22^{tm1Hsav} | MGI:3837802 | 19194496 |
| Chat | Chat^{tm1Jrs} | MGI:3045899 | 12441053 |
| Chd4 | Chd4^{tm1.1Kge} | MGI:3641408 | 15189737 |
| Cnn2 | Cnn2^{tm1.1Jin} | MGI:3820422 | 18617524 |
| Cnr1 | Cnr1^{tm1Ltz} | MGI:2182922 | 12152079 |
| Cops5 | Cops5^{tm1Rpar} | MGI:3775801 | 18268034 |
| Cops8 | Cops8^{tm1Nwe} | MGI:3762119 | 17906629 |
| Ctnnd1 | Ctnnd1^{tm1Abre} | MGI:3617486 | 16399075 |
| Ctnnd1 | Ctnnd1^{tm1Lfr} | MGI:3640772 | 16815331 |
| Cxadr | Cxadr^{tm1Know} | MGI:3815066 | 18636119 |
| Cxadr | Cxadr^{tm1Mds} | MGI:3711225 | 16543498 |
| Dab1 | Dab1^{tm1Bwh} | MGI:3777252 | 18029196 |
| Daxx | Daxx^{tm2Led} | MGI:3840084 | N/A |
| Dgat1 | Dgat1^{tm2Far} | MGI:3842432 | 19028692 |
| Dicer1 | Dicer1^{tm1Smr} | MGI:3641051 | 16099834 |
| Dsc3 | Dsc3^{tm2Pko} | MGI:3812225 | 18682494 |
| Efnb1 | Efnb1^{tm1Rha} | MGI:3653699 | 12919674 |
| Efnb1 | Efnb1^{tm1Sor} | MGI:3039289 | 15037550 |
| Efnb2 | Efnb2^{tm4Kln} | MGI:3026687 | 14699416 |
| Egln1 | Egln1^{tm2Fong} | MGI:3778917 | 16966370 |
| Egln2 | Egln2^{tm2Fong} | N/A | 16966370 |
| Egln3 | Egln3^{tm2Fong} | N/A | 16966370 |
| En1 | En1^{tm8.1Alj} | MGI:3789091 | 17537797 |
| Epb4.1/1 | Epb4.1/1^{tm1Aliv} | MGI:3838852 | 19225127 |
| Epb4.1/2 | Epb4.1/2^{tm1Aliv} | MGI:3838851 | 19225127 |
| Erap1 | Erap1^{tm1Gnie} | MGI:3830213 | 17277129 |
| Erbb4 | Erbb4^{tm1Fej} | MGI:2680217 | 12954715 |
| Erbb4 | Erbb4^{tm1Htig} | MGI:3603749 | 15863464 |
| Esrrb | Esrrb^{tm1.1Nat} | MGI:3720481 | 17765677 |
| Ets2 | Ets2^{tm4Rgo} | MGI:3769393 | 17977525 |
| Etv5 | Etv5^{tm1Sun} | N/A | 19386269 |
| Ezr | Ezr^{tm2Aim} | MGI:3052159 | 15177033 |
| F3 | F3^{tm1Nmk} | MGI:3803978 | 17663739 |
| Fgf8 | Fgf8^{tm1.1Mrt} | MGI:1857843 | 9462741 |
| Fgf9 | Fgf9^{tm1Fwan} | MGI:3621451 | 16496342 |
| Flcn | Flcn^{tm1Btt} | MGI:3829641 | 18974783 |
| Flt4 | Flt4^{tm2Ali} | MGI:3804462 | 18519586 |
| Foxd3 | Foxd3^{tm3Lby} | MGI:3790794 | 18367558 |
| Frs2 | Frs2^{tm1Fwan} | MGI:3768912 | 17868091 |
| Fzd5 | Fzd5^{tm2Nat} | MGI:3796577 | 18509025 |
| Fzr1 | Fzr1^{tm1Mama} | MGI:3800718 | 18552834 |
| Gabpa | Gabpa^{tm1Sjb} | MGI:3665312 | 17485447 |
| Gabrg2 | Gabrg2^{tm2Lusc} | MGI:2680624 | 14572465 |
| Gad1 | Gad1^{tm1Rpa} | MGI:3527168 | 17582330 |
| Gata3 | Gata3^{tm1Bchd} | MGI:3719567 | 16319112 |
| Gata3 | Gata3^{tm3Gsv} | MGI:3696958 | 17151017 |
| Gba | Gba^{tm1Clk} | MGI:3698018 | 17079175 |
| Gbx2 | Gbx2^{tm1Alj} | MGI:2388609 | 12367504 |
| Gdf1 | Gdf1^{tm1Dmus} | MGI:3806582 | 18615710 |
| Gfra1 | Gfra1^{tm2Jmi} | MGI:3715156 | 17507417 |
| Gjc1 | Gjc^{tm1Weil} | MGI:3530292 | 15659592 |
| Gli2 | Gli2^{tm6Alj} | MGI:3664541 | 16571625 |
| Gli3 | Gli3^{tm1Zllr} | N/A | this report |
| Glud1 | Glud1^{tm1.1Pma} | MGI:3835667 | 19015267 |
| Gna13 | Gna13^{tm2Cgh} | MGI:3583876 | 15919816 |
| Gpr22 | Gpr22^{tm1Jwad} | MGI:3805679 | 18539757 |
| Gpsm1 | Gpsm1^{tm1Lajb} | MGI:3807517 | 18450958 |
| Gpx4 | Gpx4^{tm2Marc} | MGI:3810783 | 18762024 |
| Grid2ip | Grid2ip^{tm1Mmsh} | MGI:3796571 | 18509461 |
| Hand1 | Hand1^{tm2Eno} | MGI:3514024 | 15576406 |
| Hand2 | Hand2^{tm1Zllr} | MGI:4453960 | 20386744 |
| Hfe | Hfe^{tm1Wsr} | MGI:3775647 | 14618243 |
| Hhex | Hhex^{tm2Cwb} | MGI:3721426 | 17580084 |
| Hoxb1 | Hoxb1^{tm7Mrc} | MGI:3046794 | 15198977 |
| Hus1 | Hus1^{tm2Rsw} | MGI:3702082 | 15919177 |
| Ift20 | Ift20^{tm1Gjp} | MGI:3817416 | 18981227 |
| Ikbkg | Ikbkg^{tm1.1Mpa} | MGI:2679024 | 10911992 |
| Insig1 | Insig1^{tm1Mbjg} | MGI:3603523 | 16100574 |
| Isl1 | Isl1^{tm2Gan} | MGI:3797783 | 18434421 |
| Itga3 | Itga3^{tm1Hap} | MGI:3833130 | 19104148 |
| Itgb1 | Itgb1^{tm3Mlkn} | MGI:3624806 | 16618804 |
| Itgb4 | Itgb4^{tm1Mfel} | MGI:3803792 | 18579745 |
| Itgb8 | Itgb8^{tm2Lfr} | MGI:3608910 | 16251442 |
| Itpr2 | Itpr2^{tm1Chen} | MGI:3640971 | 15933266 |
| Kcnj10 | Kcnj10^{tm1Kdmc} | MGI:3761690 | 17942730 |
| Klf2 | Klf2^{tm1Mlkn} | MGI:3765423 | 17141159 |
| Lama5 | Lama5^{tm2Jhm} | MGI:3612315 | 15936333 |
| Lamc1 | Lamc1^{tm1Strl} | MGI:2681365 | 14638863 |
| Ldb3 | Ldb3^{tm4Chen} | MGI:3831620 | 19028670 |
| Lims1 | Lims1^{tm1.1Chen} | MGI:3575965 | 15798193 |
| Map3k3 | Map3k3^{tm2Bisu} | MGI:3836798 | 19265138 |
| Mef2d | Mef2d^{tm3Eno} | MGI:3772400 | 18079970 |
| Mfn1 | Mfn1^{tm2Dcc} | MGI:3779080 | 17693261 |
| Mfn2 | Mfn2^{tm3Dcc} | MGI:3779081 | 17693261 |
| Mib1 | Mib1^{tm2Kong} | MGI:3804448 | 18043734 |
| Mir17-92 | Mir17-92^{tm1Tyj} | MGI:3795513 | 18329372 |
| Mll2 | Mll2^{tm1.1Afst} | MGI:3623310 | 16540515 |
| Mtmr2 | Mtmr2^{tm1Abol} | MGI:3513251 | 15557122 |
| Myb | Myb^{tm1.1Jof} | MGI:3037362 | 12941699 |
| Myd88 | Myd88^{tm1Defr} | MGI:3809600 | 18656388 |
| Myot | Myot^{tm1Moza} | MGI:3697713 | 17074808 |
| Nampt | Nampt^{tm1Oleo} | MGI:3818627 | 18802071 |
| Ncor1 | Ncor1^{tm1Anh} | MGI:3821874 | 19052228 |
| Ndufs4 | Ndufs4^{tm1Rpa} | MGI:3527173 | 18396137 |
| Neurog2 | Neurog2^{tm5(Neurog2)Fgu} | MGI:3664585 | N/A |
| Notch2 | Notch2^{tm3Grid} | MGI:3617328 | 16397869 |
| Nr5a2 | Nr5a2^{tm1Sakl} | MGI:3795276 | 18323469 |
| Nr5a2 | Nr5a2^{tm1Sjns} | MGI:3720193 | 17670946 |
| Nrp2 | Nrp2^{tm1.1Mom} | MGI:3712029 | 12019322 |
| Ntrk2 | Ntrk2^{tm2Kln} | MGI:1933974 | 10571233 |
| Numa1 | Numa1^{tm1.1Dwc} | MGI:3838102 | 19255246 |
| Numb | Numb^{tm1Ynj} | MGI:1932085 | 10841580 |
| Olig2 | Olig2^{tm1Qrlu} | MGI:3614399 | 16436615 |
| Otx2 | Otx2^{tm4.1Sia} | MGI:2178753 | 11820816 |
| Oxtr | Otxr^{tm1.1Wsy} | MGI:3800791 | 18356275 |
| Pax3 | Pax3^{tm5Buck} | MGI:3687384 | 16951257 |
| Pax6 | Pax6^{tm2Pgr} | MGI:1934348 | 11069887 |
| Pax9 | Pax9^{tm1.1Hpt} | MGI:3723638 | 17610273 |
| Paxip1 | Paxip1^{tm2Gdr} | MGI:3767658 | 17925232 |
| Pbx3 | Pbx3^{tm1Og} | MGI:3773271 | 18155191 |
| Pclo | Pclo^{tm2Sud} | MGI:3785835 | N/A |
| Pcsk5 | Pcsk5^{tm2Prat} | MGI:3789183 | 18378898 |
| Pdgfc | Pdgfc^{tm1Hdin} | MGI:3768436 | 17941048 |
| Pggt1b | Pggt1b^{tm1Mbrg} | MGI:3713756 | 17476360 |
| Pik3cb | Pik3cb^{tm1Bvan} | MGI:3795849 | 18544649 |
| Pik3r1 | Pik3r1^{tm1Lca} | MGI:3607981 | 16227599 |
| Pkd1 | Pkd1^{tm2Ggg} | MGI:3612341 | 15579506 |
| Pkd1 | Pkd1^{tm2Som} | MGI:3793791 | 18263604 |
| Pkhd1 | Pkhd1^{tm1Ggg} | MGI:3759214 | 17575307 |
| Pkp3 | Pkp3^{tm1Fvr} | MGI:3798859 | 18079750 |
| Pla2g15 | Pla2g15^{tm1Jash} | MGI:3665282 | 16880524 |
| Plec1 | Plec1^{tm4Gwi} | MGI:3721885 | 17606998 |
| Plxnb1 | Plxnb1^{tm1Ltam} | MGI:3790772 | 17519029 |
| Pofut1 | Pofut1^{tm1Ysa} | MGI:3808704 | 18547789 |
| Prss8 | Prss8^{tm1.2Hum} | MGI:2384523 | 11857812 |
| Ptch1 | Ptch1^{tm1Hahn} | MGI:3764517 | 17536012 |
| Ptger3 | Ptger3^{tm1Csml} | MGI:3764893 | 17676060 |
| Ptk2 | Ptk2^{tm1Lfr} | MGI:2684666 | 14642275 |
| Ptk2 | Ptk2^{tm1Mmsh} | MGI:3777585 | 18279360 |
| Pyy | Pyy^{tm1Batt} | MGI:3771166 | 16950139 |
| Rasa1 | Rasa1^{tm1Pdk} | MGI:3772459 | 18064675 |
| Rasgrf1 | Rasgrf1^{tm4.1Pds} | MGI:3611767 | 17030618 |
| Rela | Rela^{tm1Asba} | MGI:3775205 | 18250470 |
| Ret | Ret^{tm13Jmi} | MGI:3690534 | 17065462 |
| Ret | Ret^{tm13Jmi} | MGI:3690534 | 17065462 |
| Ret | Ret^{tm1Kln} | MGI:3662623 | 16600854 |
| Rfx3 | Rfx3^{tm1Wrth} | MGI:3045791 | 15121860 |
| Rictor | Rictor^{tm1Mgn} | MGI:3526066 | 16962829 |
| Rims1 | Rims1^{tm3Sud} | MGI:3822548 | 19074017 |
| Rtel1 | Rtel1^{tm1Hdin} | MGI:3772370 | 18064678 |
| S100a10 | S100a10^{tm1Jnw} | MGI:3665443 | 17035534 |
| Sall4 | Sall4^{tm2Tre} | MGI:3692449 | 17060609 |
| Scn1b | Scn1b^{tm2Isom} | MGI:3768513 | 17868089 |
| Scn8a | Scn8a^{tm1Mm} | MGI:3043395 | 15286995 |
| Scn9a | Scn9a^{tm1Jnw} | MGI:3053097 | 15314237 |
| Scnn1b | Scnn1b^{tm1.1Hum} | MGI:3832670 | 19036848 |
| Scnn1g | Scnn1g^{tm1.1Hum} | MGI:3832674 | 19036848 |
| Sfpi1 | Sfpi1^{tm1Dgt} | MGI:3045206 | 15146183 |
| Sfpi1 | Sfpi1^{tm1.2Nutt} | MGI:3578011 | 15867096 |
| Sh2d4a | Sh2d4a^{tm1Pdk} | MGI:3809251 | 18641339 |
| Shh | Shh^{tm2Chg} | MGI:3628824 | 16611729 |
| Slc6a9 | Slc6a9^{tm1.1Bois} | MGI:3622080 | 16554468 |
| Smad3 | Smad3^{tm1Zuk} | MGI:3822465 | 18809571 |
| Snai1 | Snai1^{tm1.1Stjw} | MGI:3838175 | 19188491 |
| Socs1 | Socs1^{tm3Wehi} | MGI:2656917 | 12705851 |
| Sox12 | Sox12^{tm1Weg} | MGI:3804456 | 18505825 |
| Sox17 | Sox17^{tm2Sjm} | MGI:3717121 | 17655922 |
| Sp6 | Sp6^{tm1Ibmm} | MGI:3778292 | 18297738 |
| Sp7 | Sp7^{tm2Crm} | MGI:3608932 | 16203988 |
| Sphk1 | Sphk1^{tm2Cgh} | MGI:3707997 | 17363629 |
| Sphk2 | Sphk2^{tm1.1Cgh} | MGI:3708000 | 17363629 |
| Spry1 | Spry1^{tm1Jdli} | MGI:3574403 | 15691764 |
| Spry2 | Spry2^{tm1Mrt} | MGI:3578632 | 15809037 |
| Spry4 | Spry4^{tm1.1Mrt} | MGI:3702553 | 16890158 |
| Supv3l1 | Supv3l1^{tm2Jkl} | MGI:3833740 | 19145458 |
| Syt9 | Syt9^{tm1Sud} | MGI:3715453 | 17521570 |
| Tbx1 | Tbx1^{tm1Dsr} | MGI:3510038 | 15469978 |
| Tcf3 | Tcf3^{tm1Mbu} | MGI:3803637 | 18538592 |
| Tex11 | Tex11^{tm1Jpt} | MGI:3797589 | 18369460 |
| Thap11 | Thap11^{tm1Tpz} | MGI:3797582 | 18585351 |
| Thoc1 | Thoc1^{tm2.1Dwg} | MGI:3698314 | 17211872 |
| Thrb | Thrb^{tm1Mkni} | MGI:3836780 | 19244534 |
| Tor1a | Tor1a^{tm2Yql} | MGI:3772564 | 17956903 |
| Tpp2 | Tpp2^{tm1Gnie} | MGI:3783749 | 18362329 |
| Traf3 | Traf3^{tm1Rbr} | MGI:3777324 | 18313334 |
| Tslp | Tslp^{tm1.1Pcn} | MGI:3837749 | 18650845 |
| Ttn | Ttn^{tm1Her} | MGI:2651645 | 12464612 |
| Txnrd1 | Txnrd1^{tm1Marc} | MGI:3574358 | 15713651 |
| Txnrd2 | Txnrd2^{tm1Marc} | MGI:3512408 | 15485910 |
| Uba7 | Uba7^{tm1Dzh} | MGI:3521787 | 16382139 |
| Upf2 | Upf2^{tm1Btp} | MGI:3790198 | 18483223 |
| Vcl | Vcl^{tm1Ross} | MGI:3769142 | 17785437 |
| Vprbp | Vprbp^{tm1.1Yxi} | MGI:3814062 | 18606781 |
| Wnt3 | Wnt3^{tm2Amc} | MGI:2450903 | 12569130 |
| Wnt7b | WntTb^{tm1Amc} | MGI:3526431 | 16163358 |
| Wnt9a | Wnt9a^{tm1Chha} | MGI:3701348 | 16818445 |
| Yy1 | Yy1^{tm2Yshi} | MGI:3625967 | 16611997 |

b. dRMCE Strategy

Conditional loss-of-function Hand2 allele (Hand2^{f} or also designated Hand2^{fneo}) have previously been generated by HR, which involved analysis of over 500 colonies to identify one correctly targeted mES cell clone (1/576; 0.17% HR targeting frequency as reported previously (Srivastava et al. (1997) Nat. Genet. 16, 154-60). G418 resistant ES-cell clones (576 in total) were screened by Southern blot analysis. One ES-cell clone (4D7) was fully recombined and germ-line transmission from chimeric mice was obtained (Galli A. et al. (2010) PLoS Genet 6(4): e1000901); FIG. 2a.

In an attempt to introduce a FLAG epitope into the endogenous HAND2 protein without the need to use HR again, the following dRMCE strategy was developed (FIG. 2a). First, a replacement vector (FIG. 2a) was constructed in which the modified FLAG-tagged Hand2 locus is flanked by single loxP and FRT sites in the same orientation as in the Hand2^{f} locus. Furthermore, the PGK-hygromycin selection cassette was inserted downstream of the second Hand2 coding exon and flanked by φC31 target sites (Belteki et al. (2003) Nat. Biotechnol. 21, 321-4) to enable its removal in correctly recombined mES cell clones. Second, the pDIRE expression plasmid (FIG. 3) was developed to enable efficient co-expression of the optimized iCRE and FLPo recombinases (Shimshek et al. (2002) Genesis 32, 19-26; Raymond, C. S. & Soriano, P. (2007) PLoS ONE 2, e162) in target mES cells. Simultaneous transfection of the replacement vector and expression of both recombinases is key, as the conditional Hand2^{fneo} locus could potentially undergo extensive rearrangements due to the presence of two loxP and FRT sites. As several undesired genomic recombination events and rearrangements are possible in addition to correct replacement, a panel of primer pairs was designed to discriminate correct from incomplete or aberrant recombination events (Table 1).

Following co-transfection of the replacement and pDIRE vectors into Hand2^{f} heterozygous mES cells, hygromycin-resistant colonies were selected and screened by PCR to detect replacement events. 54 of 343 mES cell clones displayed PCR fragment patterns indicative of correct replacement (FIGS. 2a, 2b, and Table 3).

TABLE 3
Frequencies of the different recombination events detected
at the conditional Hand2 locus following dRMCE
Dual-RMCE Hand2 targeting:

| Positive for: | N | % |
|---|---|---|
| Hand2^{FLAG} (5′ and 3′ screening) | 43 | 12.54 |
| Hand2^{FLAG}/Hand2^{Δ} (mixed clones) | 11 | 03.21 |
| Hand2^{Δ} | 131 | 38.19 |
| Hand2^{FLAG} neg./Hand2^{Δ} neg. | 158 | 46.06 |
| Total clones picked | 343 | |

12.54% of all clones are heterozygous for the Hand2^{Flag-hygro} allele. Further analysis revealed that 11 clones displayed patterns indicative of the simultaneous presence of the replacement (i.e. Hand2^{FLAG}) and the deleted (Hand2^{Δ}) alleles. As the parental mES cells are heterozygous for the Hand2^{f} allele, these “clones” must represent a mixed population of cells having undergone recombination. This unusually high proportion of mixed clones (20% of all the positive clones) is unlikely to arise by cross-contamination during picking, but rather indicates that cis-recombination is favoured (Zheng et al. (2000) Mol. Cell Biol. 20, 648-55) and occurs prior to recombination with the Hand2^{FLAG} replacement cassette. These mixed clones can be easily explained if deletion of the genomic region occurs before cell division, followed by unequal segregation and recombination of the replacement vector in e.g. only one of the daughter cells. Consistent with this interpretation, cis-recombination (i.e. appearance of the Hand2^{Δ} allele) is accompanied by random integration of the replacement vector in a very large fraction of all hygromycin resistant clones (131/343, see Table 3).

In summary, 12.5% (43/343) of all mES cell clones isolated have undergone complete and correct replacement of the Hand2^{f} with the Hand2^{FLAG} allele. This is about 70-fold more efficient than conventional HR at the Hand2 locus (1 correct clone out of 576, Galli A. et al. (2010) PLoS Genet 6(4): e1000901).

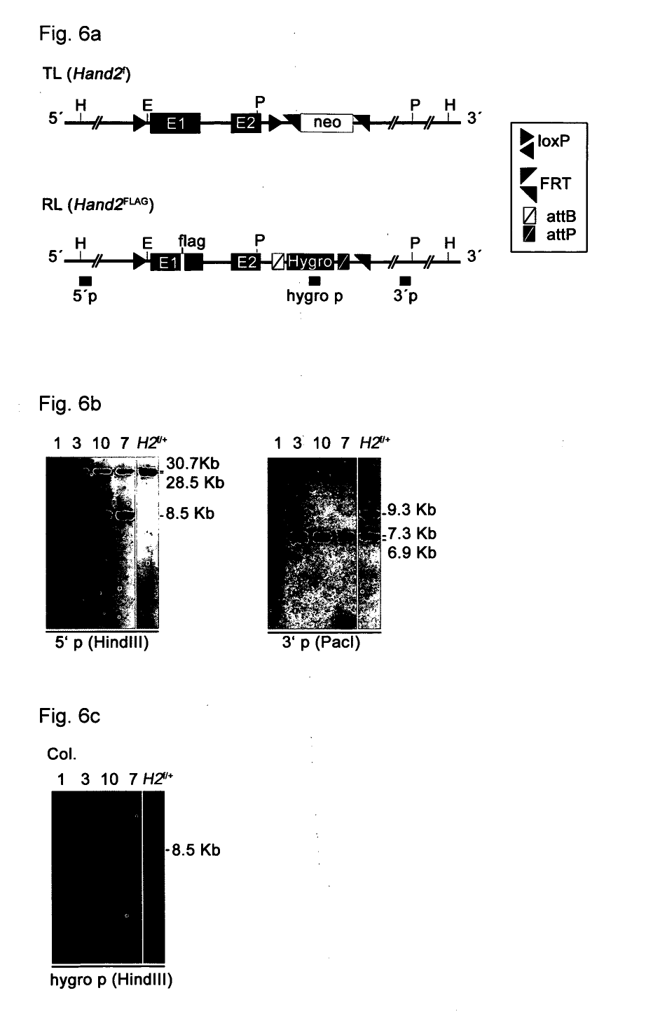

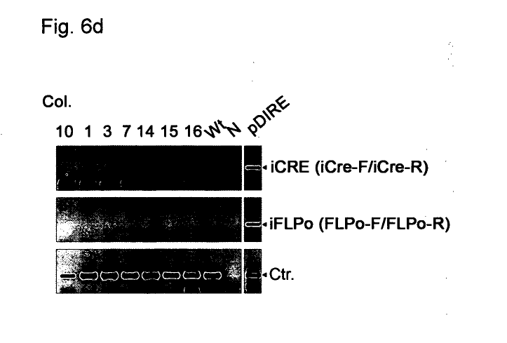

Four of the Hand2^{FLAG} clones were subjected to Southern blot analysis, which revealed the genomic integrity of the Hand2 locus with the inserted FLAG tag and ruled out random integration of additional hygromycin-resistance cassettes and the pDIRE vector (FIG. 6). These clones were also transiently electroporated with a φC31 expression vector to delete the hygromycin selection cassette, which can also be removed in mice using the available φC31 “deleter” mouse strain (Raymond, C. S. & Soriano, P. (2007) PLoS ONE 2, e162). This is important as the PGK promoter (driving hygromycin expression, FIG. 2a) might alter the expression of the endogenous locus, as shown for many other conditional alleles (Meyers et al. (1998) Nat. Genet. 18, 136-41). Finally, two correctly engineered Hand2^{FLAG} mES cell clones (14B6 and 14B2) were injected into mouse blastocysts, which resulted in efficient production of several highly chimeric mice. Chimeric males from both clones transmitted Hand2^{FLAG} allele to their F1 progeny (FIG. 2c), which do not display any phenotypic abnormalities. These results establish that the entire dRMCE procedure does not alter the germline transmission potential of mES cells nor cause abundant chromosomal abnormalities and phenotypic effects.

The general potential of the dRMCE technology was evidenced by targeting a completely unrelated heterologous locus. The recently generated Gli3^{neo} allele was chosen as it encodes a dRMCE compatible configuration of loxP and FRT sites. Following transfection of Gli3^{neo/+} mES cells with the Hand2^{FLAG} and pDIRE vectors, 113 hygromycin resistant clones were screened by PCR, which revealed correct insertion of Hand2^{FLAG} into the Gli3 locus in 37 mES cell clones (32.7%, Table 4).

TABLE 4
Frequencies of the different recombination events detected
at the heterologous Gli3 locus following dRMCE
Dual-RMCE Gli3 targeting

| Positive for: | N | % |
|---|---|---|
| Gli3^{Hand2FLAG} (5′/3′ screening) | 37 | 32.74 |
| Gli3^{Δneo} | 28 | 24.78 |
| Gli3^{neo} | 48 | 42.48 |
| Total clones picked | 113 | |

32.74% of all clones are heterozygous for the inserted Hand2Flag-hygro allele. Conventional RMCE with heterotypic loxP sites and an unrelated replacement vector at this locus resulted in a replacement frequency of 21% (27/130), which indicates that the efficiency of dRMCE comparable to conventional RMCE. Finally, a dRMCE toolkit was constructed that consists of the pDIRE plasmid (FIG. 3a) and four pDRAV targeting plasmids in which loxP and FRT sites are present in all possible orientations (FIG. 3b) to allow easy insertion of any replacement cassette. This dRMCE toolkit will allow replacement-type engineering of a large number of available mouse mutant alleles. The MGI database (http://www.informatics.jax.org) shows over 200 conditional alleles compatible with the inventive dRMCE technology (see Table 2).

### Smad4

The Smad4 knockout-first allele (Smad4^{tm1a(EUCOMM)Wtsi}, denoted here as Smad4^{f}) contains a promoterless gene trap selection cassette (lacZ-T2A-neo) flanked by FRT sites and followed by a critical exon flanked by loxP sites. Heterozygous C57BU6 Smad4^{f} ES cells were co-transfected with pDIRE and pDREV-1 vectors, the latter encoding a H2B-Venus YFP reporter (FIG. 4a). Puromycin-resistant colonies were screened by short-range PCR at the 3′ (loxP) and the 5′ (FRT) junctions for correct replacement events, which generate the YFP-tagged Smad4 allele (Smad4^{YFP}; FIG. 4b). Indeed, most ES colonies analyzed are correctly replaced and of clonal origin (69%:33 out of 48 clones, see Table 5). These mixed colonies are easily recognized and likely arise as a consequence of partial recombination events. Therefore, correct replacement by dRMCE must always be validated by confirming the absence of both the floxed and deleted allele.

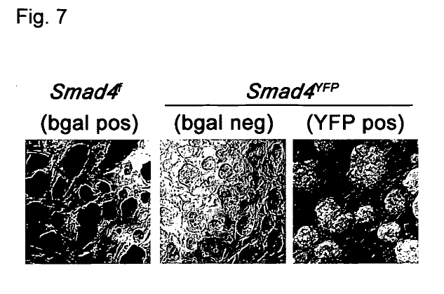

Furthermore, correctly recombined clones by dRMCE have undergone the substitution of the lacZ reporter (encoding beta-galactosidase) by the H2B-Venus reporter (encoding a yellow fluorescent protein). Indeed, direct detection of the fluorescent reporter serves to monitor recombination by dRMCE and therefore can also be used to identify correctly recombined clones even in the absence of a drug-selection step (FIG. 7).

### Zfp503

The Zfp503^{f} promoter-driven knockout-first allele (Zfp503^{tm1a(KOMP)Wtsi}) encodes three loxP and two FRT sites (FIG. 5a). Recipient Zfp503f/+ ES cells were co-transfected with pDIRE and pDREV-0 plasmids and colonies were selected using puromycin. Generation of the correctly replaced Zfp503^{YFP} allele was again highly efficient (52%, see FIG. 5b and Table 5) and will lead to expression of the YFP reporter under the control of the endogenous locus.

TABLE 5
Frequencies of correct targeting of IKMC conditional alleles by dRMCE
Dual-RMCE Smad4 and Zfp503 targeting frequencies

| | Colonies | Correct | | |
|---|---|---|---|---|
| Gene | analyzed | dRMCE | Mixed | Negative |
| Smad4 | 48 | 33 (69%) | 5 | 10 |
| Zfp503 | 48 | 25 (52%) | 0 | 23 |

69% and 52% of all clones are correctly recombined after dRMCE in the Smad4 and Zfp503 loci, respectively.

dRMCE allows re-engineering of conventional targeted alleles with frequencies of up to ˜70% correct replacement. Minimally, this represents a 5 to 65-fold increase in efficiency in comparison to HR (Table 6), which is of particular benefit for difficult to target loci. Even at the lowest efficiency observed (13% for Hand2), very few colonies need to be analyzed to identify correctly replaced clones.

TABLE 6
Targeting frequencies by dRMCE compared to those obtained
by homologous recombination (HR) at the same loci.

| | | Homologous | |
|---|---|---|---|
| Gene | dRMCE | recombination | Fold increase |
| Hand2 | 13% | 0.2% | 65x |
| Gli3 | 33% | 3% | 11x |
| Smad4 | 69% | 3-6% | 12x |
| Zfp503 | 52% | 11% | 5x |

1. A method for integrating a DNA of interest into an embryonic rodent stem cell having a conditional allele containing a first and a second recombination site, which are not identical and not recognized by the same recombinase, comprising
(a) introducing into said embryonic rodent stem cell a first and a second recombinase specific for the first and for the second recombination site, respectively;
(b) introducing into said embryonic rodent stem cell a targeting vector comprising a vector cassette that encodes said DNA of interest flanked by the first and the second recombination site, and
(c) identifying and isolating embryonic rodent stem cells wherein the conditional allele is replaced with the DNA of interest.

2. The method of claim 1 wherein the embryonic stem cell is a mouse embryonic stem cell.

3. The method of claim 1 wherein in step (a) the first and the second recombinase are introduced by introducing a fragment of DNA capable of expressing the first and the second recombinase.

4. The method of claim 1 wherein in step (a) the first and the second recombinase are introduced by introducing a plasmid or plasmids capable of expressing the first and the second recombinase.

5. The method of claim 1 wherein one of the recombination sites can be recombined by a CRE recombinase or an active variant thereof.

6. The method of claim 1 wherein one of the recombination sites can be recombined by a FLP recombinase or an active variant thereof.

7. The method of claim 1 wherein the first and the second recombination site are selected from the group consisting of loxP, lox71, lox66, lox511, lox5171, lox2272, lox2722, m2, and L1, and the group consisting of FRT, F3, F5, f2161, f2151, f2262, and f61, respectively.

8. The method of claim 7 wherein the first and the second recombination site are loxP and FRT, respectively.

9. The method of claim 1 wherein the fragment of DNA capable of expressing a first and a second recombinase expresses iCRE and FLPo.

10. The method of claim 9 wherein the plasmid comprising the fragment of DNA capable of expressing a first and a second recombinase is pDIRE.

11. The method of claim 1 wherein in step (c) identification of correctly recombined embryonic mouse stem cells is based on properties of the replaced allele and/or the properties of the newly introduced DNA expression product.

12. The method of claim 1 wherein in step (b) the vector cassette comprises a DNA encoding a selection marker and in step (c) identification is done selecting transfected cells expressing the selection marker.

13. The method of claim 12 wherein the targeting vector for step (b) is generated u plasmids pDRAV-1, pDRAV-2, pDRAV-3 and/or pDRAV-4.

14. The method of claim 1 wherein the fragment of DNA of step (a) and the vector of step (b) are introduced simultaneously.

15. The method of claim 1, wherein libraries of embryonic stem cells carrying single or multiple point mutations, single or multiple deletions or insertion of desired DNA into the locus of the conditional allele are generated.

16. A kit for integrating a DNA of interest into rodent embryonic stem cells carrying a conditional allele comprising (a) a fragment of DNA capable of expressing a first and a second recombinase specific for the first and for the second recombination site, respectively; and (b) a targeting vector comprising a vector cassette that encodes a DNA of interest flanked by the first and the second recombination site.

17. The kit of claim 16 wherein the fragment of DNA expresses CRE or an active variant thereof and FLP or an active variant thereof.

18. The kit of claim 17 wherein the fragment of DNA expresses iCRE and FLPo.

19. The kit of claim 18 wherein the plasmid comprising the fragment of DNA is pDIRE.

20. The kit of claim 16 wherein the first and the second recombination site on the targeting vector is loxP and FRT.

21. The kit of claim 16 further comprising plasmids pDRAV-1, pDRAV2, pDRAV-3 and/or pDRAV-4.
